(19) United States
(12) Patent Application Publication
(10) Pub. No.: US 2005/0079142 A1
(43) Pub. Date: Apr. 14, 2005

(54) Use of UV filter substances for optimizing the quality of cosmetic foams

(75) Inventors: Melanie Brunckhorst, Hamburg (DE); Anja Eitrich, Hamburg (DE); Kerstin Hoop, Pinneberg (DE); Claudia Mundt, Bremen (DE); Ilka Oelrichs, Tornesch (DE); Heidi Riedel, Hamburg (DE); Stefan Scheede, Hamburg (DE); Rixa Von Bulow, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/922,644

(22) Filed: Aug. 19, 2004

(30) Foreign Application Priority Data
Aug. 19, 2003 (DE) 103 38 012.4

(51) Int. Cl. A61K 7/42; A61K 7/15

(52) U.S. Cl. 424/59

(57) ABSTRACT

The present invention is a self-foaming, foam-like, after-foaming or foamable cosmetic or dermatological preparation comprising one or more UV filter substances that boost the foam, reduce the bubble size and/or increase the bubble number of the preparation. The present invention also includes methods of boosting the foam or reducing the bubble size of a cosmetic or dermatological preparation.

### FIELD OF THE INVENTION

The present invention relates to the use of UV filter substances for optimizing the quality of self-foaming, foam-like, after-foaming or foamable cosmetic and dermatological preparations.

### BACKGROUND OF THE INVENTION

Foams or foam-like preparations are a type of disperse system.

By far the most important and best known disperse system are emulsions. Emulsions are two- or multi-phase systems of two or more liquids which are insoluble or only slightly soluble in one another. The liquids (pure or as solutions) are present in an emulsion in a more or less fine distribution, which generally has only limited stability.

Foams are structures of gas-filled, spherical or polyhedral cells which are delimited by liquid, semiliquid, high-viscosity or solid cell ribs. The cell ribs, connected via points of intersection, form a continuous framework. The foam lamellae stretch between the cell ribs (closed-cell foam). If the foam lamellae are disturbed or if they flow back into the cell rib at the end of foam formation, an open-cell foam is obtained. Foams are also thermodynamically unstable since a reduction in the surface area leads to the production of surface energy. The stability and thus the existence of a foam is thus dependent on the extent to which it is possible to prevent its self-destruction.

Cosmetic foams are usually dispersed systems of liquids and gases, where the liquid represents the dispersant and the gas represents the dispersed substance. Foams of low-viscosity liquids are temporarily stabilized by surface-active substances (surfactants, foam stabilizers). Because of their large internal surface area, such surfactant foams have a high adsorption capacity, which is utilized, for example, in cleaning and washing operations. Accordingly, cosmetic foams are used, in particular, in the fields of cleansing, for example as shaving foam, and of haircare.

To generate foam, gas is bubbled into suitable liquids, or foam formation is achieved by vigorously beating, shaking, spraying or stirring the liquid in the gas atmosphere in question, provided that the liquids comprise suitable surfactants or other interface-active substances (“foam formers”), which, apart from interfacial activity, also have a certain film-forming ability.

Cosmetic foams have the advantage over other cosmetic preparations of permitting a fine distribution of active ingredients on the skin. However, cosmetic foams can generally only be achieved using particular surfactants, which, moreover, are often not well tolerated by the skin.

A disadvantage of the prior art is that such foams have only low stability, for which reason they usually collapse within approximately 24 hours. A requirement of cosmetic preparations, however, is that they have stability for years, as far as possible. This problem is generally taken into account by the fact that the consumer produces the actual foam himself just before use using a suitable spray system, for which purpose, for example, it is possible to use spray cans in which a liquefied pressurized gas serves as propellent gas. Upon opening the pressure valve, the propellent liquid mixture escapes through a fine nozzle, and the propellant evaporates, leaving behind a foam.

After-foaming cosmetic preparations are also known per se. They are firstly applied to the skin from an aerosol container in flowable form and, after a short delay, develop the actual foam only once they are on the skin under the effect of the after-foaming agent present, for example a shaving foam. After-foaming preparations are often in specific formulation forms, such as, for example, after-foaming shaving gels or the like.

The prior art also recognizes cosmetic or dermatological preparations which are already foamed during the preparation and have sufficiently high stability per se in order to be packaged, stored and traded in the usual manner (see, for example, DE-100 63 340-A1, DE-100 63 341-A1 and DE-100 63 342-A1).

However, a disadvantage of the prior art preparations is often the quality of the foam, i.e. the foam is soft, coarsely-bubbled and, following removal from the container is only slightly stable. Also, foamable cosmetic emulsions (so-called “aerosol foams”) cannot be formulated or be prepared industrially in accordance with the prior art without the use of particular surfactants. This is true in particular for systems which are based on classical emulsifiers. According to the prior art, such systems, when propellent gas is added, develop exclusively aqueous-moist foams which rapidly break following application.

### SUMMARY OF THE INVENTION

The object of the present invention was therefore to enrich the prior art and to provide cosmetic or dermatological self-foaming and/or foam-like preparations which do not have the disadvantages of the prior art.

It was surprising and could not have been foreseen by the person skilled in the art that the use of one or more UV filter substances for the foam-boosting of self-foaming, foam-like, after-foaming or foamable cosmetic and dermatological preparations overcomes the disadvantages of the prior art.

### BRIEF DESCRIPTION OF THE DRAWINGS

FIG. 1 is a photograph of a cosmetic or dermatological preparation comprising a polar oil according to the Comparative Example.

FIG. 2 is a photograph of a cosmetic or dermatological preparation comprising a polar oil and at least one UV filter substance according to Example 1.

### DETAILED DESCRIPTION OF THE PREFERRED EMBODIMENTS

For the purposes of the present invention, “self-foaming”, “foam-like”, “after-foaming” and “foamable” are understood as meaning that the gas bubbles are present in (any) distributed form in one (or more) liquid phase(s), the preparations not necessarily having to have the appearance of a foam in macroscopic terms. Cosmetic or dermatological preparations for the purposes of the present invention (for the sake of simplicity also referred to below as foams) may, for example, be macroscopically visibly dispersed systems of gases dispersed in liquids. The foam character can, however, for example, be visible only under a (light) microscope. Moreover, foams according to the present invention—particularly when the gas bubbles are too small to be seen under a light microscope—are also recognizable from the sharp increase in the volume of the system.

As a result of the invention, a rich, compact cream foam (“mousse”) is accessible for the first time, which is characterized by a long storage period, an extraordinarily high stability, and a compact appearance. Compositions according to the invention develop into finely-bubbled foams even during their manufacture—for example during stirring or during homogenization. According to the invention, finely-bubbled, rich foams of excellent cosmetic elegance are obtainable. Furthermore, preparations which are particularly well tolerated by the skin are obtainable according to the invention, it being possible to distribute valuable ingredients particularly well on the skin.

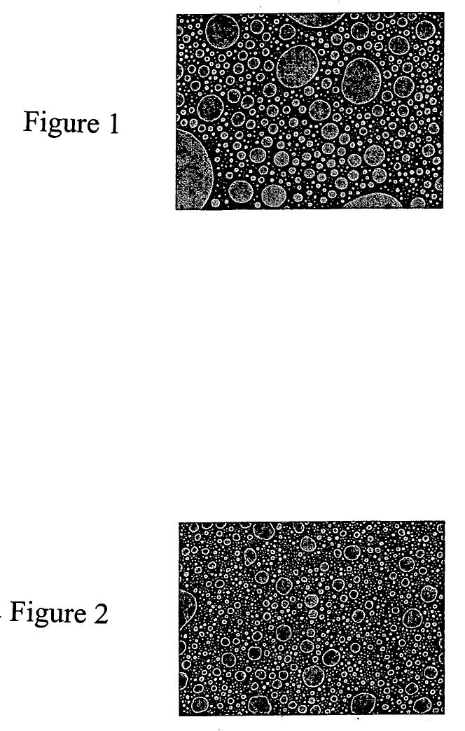

The quality of foams which comprise polar oils (such as those illustrated in FIG. 1) is poorer compared with that of foams which comprise only nonpolar oils: the foam is softer, more coarsely-bubbled and less stable.

Surprisingly, as shown in FIG. 2, the foam quality of foams comprising polar oils is significantly better when, in addition to polar oil, at least one UV filter substance is also used: the foam is compact, finely-bubbled, stable and creamy.

The invention therefore also provides for the use of one or more UV filter substances for the foam-boosting of self-foaming, foam-like, after-foaming or foamable cosmetic and dermatological preparations which comprise at least one polar oil component.

Moreover, “foam boosting” means that the stability of the foamed preparations (the “foam stability”), when compared with otherwise identical preparations that do not comprise UV filter substances according to the invention, is significantly improved. That is, through the use according to the invention, breakage of the foams is delayed.

In addition, “foam boosting” for the purposes of the present invention is also understood as meaning that the cosmetic properties of the foams according to the invention are significantly improved compared with preparations that do not comprise UV filter substances according to the invention. Thus, as a result of the use according to the invention, rich, solid foams (“foam creams”) are obtained which, despite their compactness and richness, can be spread easily and absorbed rapidly.

In particular, for the purposes of the present invention, “foam boosting” is understood as meaning that the bubble size of the foams according to the invention is smaller than that of preparations which do not comprise UV filter substances according to the invention. By contrast, the number of bubbles in the same volume is increased.

For the purposes of the present invention, “bubbles” means the small gas bubbles, and “bubble size” is accordingly understood as meaning the diameter of the small gas bubbles.

This effect can be explained very easily by reference to a microscopic foam investigation. The aim of this investigation is the microscopic determination of the number and size of the bubbles in cosmetic products.

A microscopic foam investigation can, for example, be carried out as follows at a constant temperature of 21° C.:
Aerosol foams are sprayed into a reservoir or onto cellulose, and the samples are drawn from the reservoir using a spatula from three different points (for example top, middle, bottom) of the cosmetic preparation to be investigated from the container (corresponding to three samples per preparation).

The samples, initially weighing 9 mg±0.5 mg, are placed on microscope slides
The sample is covered with a sight glass
prior to the actual recording, a 20 g weight is placed on the microscope slide with sight glass for 15 sec
three images are then recorded for each sample, the investigated area being 6.22 mm×4.66 mm=28.11 mm². For this purpose, the microscope is adjusted to 125 times magnification and the sample is illuminated in transmitted light (digital camera Polaroid DMC and image analysis programme KS400).

Advantageous preparations for the purposes of the present invention have an average bubble size of from 0.005 to 0.5 mm², advantageously from 0.005 to 0.2 mm², particularly advantageously from 0.005 to 0.1 mm², and a bubble number of from 100 to 4000, advantageously from 500 to 3000, particularly advantageously from 1000 to 3000, in each case based on the image section under analysis (see above).

Advantageous UV filter substances for the purposes of the present invention are those given below, which may be present in the water phase and/or the oil phase.

The preparations according to the invention can advantageously also be in the form of so-called oil-free cosmetic or dermatological emulsions which comprise a water phase and at least one UV filter substance which is liquid at room temperature as a further phase.

Particularly advantageous UV filter substances which are liquid at room temperature for the purposes of the present invention are 2-ethylhexyl 2-cyano-3,3-diphenylacrylate (INCI: Octocrylene) and esters of cinnamic acid, preferably 2-ethylhexyl 4-methoxycinnamate (INCI: Octyl Methoxycinnamate) and isopentyl 4-methoxycinnamate (INCI: Isoamyl p-Methoxycinnamate), 3-(4-(2,2-bis-ethoxycarbonylvinyl)phenoxy)propenyl)methoxysiloxane/di-methylsiloxane copolymer, which is available, for example, under the trade name Parsol® SLX from Hoffmann La Roche.

Advantageous UV-A filter substances for the purposes of the present invention are dibenzoylmethane derivatives, in particular 4-(tert-butyl)-4′-methoxydibenzoylmethane (CAS No. 70356-09-1), which is sold by Givaudan under the name Parsol® 1789 and by Merck under the trade name Eusolex® 9020.

Further advantageous UV-A filter substances for the purposes of the present invention are hydroxybenzophenones which are characterized by the following structural formula: 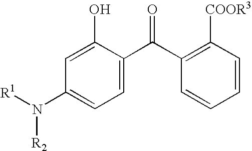
in which
R¹ and R², independently of one another, are hydrogen, C₁-C₂₀-alkyl, C₃-C₁₀-cycloalkyl or C₃-C₁₀-cycloalkenyl, where the substituents R¹ and R² together with the nitrogen atom to which they are bonded, can form a 5-membered or 6-membered ring, and
R³ is a C₁-C₂₀-alkyl radical.

A particularly advantageous hydroxybenzophenone for the purposes of the present invention is hexyl 2-(4′-diethylamino-2′-hydroxybenzoyl)benzoate (also: Aminobenzophenone), which is characterized by the following structure: 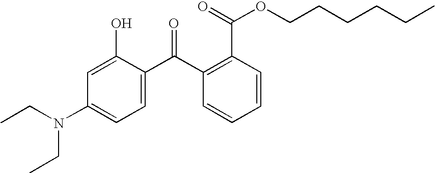
and is available under the trade name Uvinul A Plus from BASF.

Also preferred according to the invention is 2,4-bis[5-1(dimethylpropyl)benzoxazol-2-yl-(4-phenyl)imino]-6-(2-ethylhexyl)imino-1,3,5-triazine with the CAS No. 288254-16-0, which is characterized by the structural formula 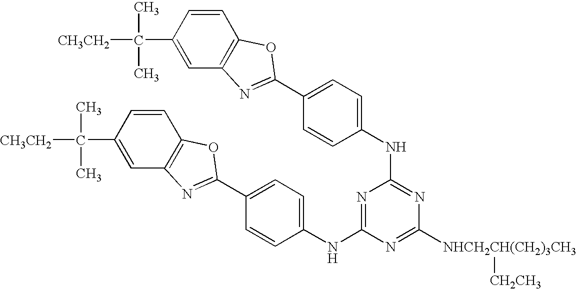
and is available from 3V Sigma under the trade name Uvasorb® K2A.

The 2,4-bis[5-1(dimethylpropyl)benzoxazol-2-yl-(4-phenyl)imino]-6-(2-ethylhexyl)imino-1,3,5-triazine is advantageously present in dissolved form in the cosmetic preparations according to the invention. In some instances, however, it may also be advantageous when the 2,4-bis[5-1(dimethylpropyl)benzoxazol-2-yl-(4-phenyl)imino]-6-(2-ethylhexyl)imino-1,3,5-triazine is present in pigmentary form, i.e. undissolved form—for example in particle sizes of 10 nm to 300 nm.

The total amount of 2,4-bis[5-1(dimethylpropyl)benzoxazol-2-yl-(4-phenyl)imino]-6-(2-ethylhexyl)imino-1,3,5-triazine in the finished cosmetic or dermatological preparations is advantageously chosen from the range 0.01% by weight to 20% by weight, preferably from 0.1 to 10% by weight, in each case based on the total weight of the preparations.

Other advantageous UV filter substances for the purposes of the present invention are sulphonated, water-soluble UV filters, such as, for example:
phenylene-1,4-bis(2-benzimidazyl)-3,3′-5,5′-tetrasulphonic acid and its salts, particularly the corresponding sodium, potassium or triethanolammonium salts, in particular the phenylene-1,4-bis(2-benzimidazyl)-3,3′-5,5′-tetrasulphonic acid bis-sodium salt with the INCI name Bisimidazylate (CAS No.: 180898-37-7), which is available, for example, under the trade name Neo Heliopan AP from Haarmann & Reimer;
salts of 2-phenylbenzimidazole-5-sulphonic acid, such as its sodium, potassium or triethanolammonium salt, and the sulphonic acid itself with the INCI name Phenylbenzimidazole Sulphonic Acid (CAS No. 27503-81-7), which is available under the trade name Eusolex 232 from Merck, or under Neo Heliopan Hydro from Haarmann & Reimer;
1,4-di(2-oxo-10-sulpho-3-bornylidenemethyl)benzene (also 3,3′-(1,4-phenylenedimethylene)bis(7,7-dimethyl-2-oxobicyclo[2.2.1]-hept-1-ylmethanesulphonic acid) and salts thereof (particularly the corresponding 10-sulphato compounds, in particular the corresponding sodium, potassium or triethanolammonium salt), which is also referred to as benzene-1,4-di(2-oxo-3-bornylidenemethyl-10-sulphonic acid). Benzene-1,4-di(2-oxo-3-bornylidenemethyl-10-sulphonic acid) has the INCI name Terephthalidene Dicamphor Sulphonic Acid (CAS No.: 90457-82-2) and is available, for example, under the trade name Mexoryl SX from Chimex;
sulphonic acid derivatives of 3-benzylidenecamphor, such as, for example, 4-(2-oxo-3-bornylidenemethyl)benzenesulphonic acid, 2-methyl-5-(2-oxo-3-bornylidenemethyl)sulphonic acid and salts thereof.

Advantageous UV filter substances for the purposes of the present invention are also so-called “broadband filters,” i.e. filter substances which absorb both UV-A and also UV-B radiation.

Advantageous broadband filters or UV-B filter substances are, for example, triazine derivatives, such as, for example,
dioctylbutylamidotriazone (INCI: Diethylhexyl Butamido Triazone), which is available under the trade name UVASORB HEB from 3V Sigma;
tris(2-ethylhexyl) 4,4′,4″-(1,3,5-triazine-2,4,6-triyltriimino)tris-benzoate, also 2,4,6-tris[anilino(p-carbo-2′-ethyl-1′-hexyloxy)]-1,3,5-triazine (INCI: Octyl Triazone), which is sold by BASF Aktiengesellschaft under the trade name UVINUL® T 150.

Bis-resorcinyltriazine derivatives are also advantageous UV filter substances for the purposes of the present invention. They are characterized by the following structural formula: 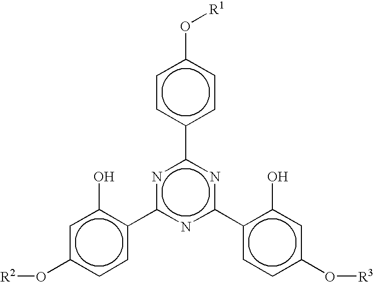
where R¹, R² and R³, independently of one another, are either hydrogen atoms or chosen from the group of branched and unbranched alkyl groups having 1 to 10 carbon atoms. According to the invention, particular preference is given to 2,4-bis{[4-(2-ethylhexyloxy)-2-hydroxy]phenyl}-6-(4-methoxyphenyl)-1,3,5-triazine (INCI: Bis-Ethylhexyloxylphenol Methoxyphenyl Triazine), which is available under the trade name Tinosorbe S from CIBA-Chemikalien GmbH. A further preferred bis-resorcinyltriazine derivative is 2-[4,6-bis(2,4-dimethylphenyl)-1,3,5-triazin-2-yl]-5-(octyloxy)phenol (CAS No.: 2725-22-6).

The total amount of one or more bis-resorcinyltriazine derivatives in the finished cosmetic or dermatological preparations is advantageously chosen from the range 0.01% by weight to 20% by weight, preferably from 0.1 to 10% by weight, in each case based on the total weight of the preparations.

An advantageous broadband filter for the purposes of the present invention is also 2,2′-methylenebis(6-(2H-benzotriazol-2-yl)-4-(1,1,3,3-tetramethylbutyl)phenol), which is available under the trade name Tinosorb® M from CIBA-Chemikalien GmbH.

For the purposes of the present invention, an advantageous broadband filter is also 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) with the INCI name Drometrizole Trisiloxane.

The further UV filter substances may be oil-soluble or water-soluble. Advantageous oil-soluble filter substances are, for example:
3-benzylidenecamphor derivatives, preferably 3-(4-methylbenzylidene)camphor, 3-benzylidenecamphor;
4-aminobenzoic acid derivatives, preferably 2-ethylhexyl 4-(dimethylamino)benzoate, amyl 4-(dimethylamino)benzoate;
2,4,6-trianilino(p-carbo-2′-ethyl-1′-hexyloxy)-1,3,5-triazine;
esters of benzalmalonic acid, preferably di(2-ethylhexyl)-4-methoxybenzalmalonate;
esters of cinnamic acid, preferably 2-ethylhexyl 4-methoxycinnamate, isopentyl 4-methoxycinnamate;
derivatives of benzophenone, preferably 2-hydroxy-4-methoxybenzophenone, 2-hydroxy-4-methoxy-4′-methylbenzo-phenone, 2,2′-dihydroxy-4-methoxybenzophenone and
UV filters bonded to polymers.

Particularly advantageous UV filter substances are:
phenylene-1,4-bis(2-benzimidazyl)-3,3′-5,5′-tetrasulphonic acid bis-sodium salt (INCI: Bisimidazylate, trade name: Neoheliopan AP),
dioctylbutylamidotriazone (INCI: Diethylhexyl Butamido Triazone; trade name: Uvasorb HEB),
2,4-bis[5-1 (dimethylpropyl)benzoxazol-2-yl-(4-phenyl)imino]-6-(2-ethylhexyl)imino-1,3,5-triazine (trade name: Uvasorb K2A),
hexyl 2-(4′-diethylamino-2′-hydoxybenzoyl)benzoate (also: Aminobenzophenone) (INCI: Diethylamino Hydroxybenzoyl Hexyl Benzoate; trade name: Uvinul A plus),
(3Z)-1,7,7-trimethyl-3-(4-methylbenzylidene)bicyclo[2.2.1]heptan-2-one (INCI: 4-Methylbenzylidene Camphor; trade name: Eusolex 6300),
2-ethylhexyl 2-cyano-3,3-diphenylacrylate (INCI: Octocrylene, trade name: Uvinul N-539),
benzene-1,4-di(2-oxo-3-bornylidenemethyl-10-sulphonic acid) (INCI: Terephtalidene Dicamphor Sulphonic Acid; trade name: Mexoryl SX),
2-(2H-benzotriazol-2-yl)-4-methyl-6-[2-methyl-3-[1,3,3,3-tetramethyl-1-[(trimethylsilyl)oxy]disiloxanyl]propyl]phenol (INCI: Drometrizole Trisiloxane; trade name: Mexoryl XL).

Very particularly advantageous UV filter substances for the purposes of the present invention are:
2,4,6-tris[anilino(p-carbo-2′-ethyl-1′-hexyloxy)]-1,3,5-triazine (INCI: Octyl Triazone; trade name: Uvinul T150),
2-ethylhexyl 4-methoxycinnamate (INCI: Octyl Methoxycinnamate; trade name: Parsol MCX),
4-(tert-butyl)-4′-methoxydibenzoylmethane (INCI: Butylmethoxydibenzoylmethane; trade name: Parsol 1789),
2,4-bis{[4-(2-ethylhexyloxy)-2-hydroxy]phenyl}-6-(4-methoxyphenyl)-1,3,5-triazine (INCI: Bis-Ethylhexyloxylphenol Methoxyphenyl Triazine; trade name: Tinosorb S),
salts of 2-phenylbenzimidazole-5-sulphonic acid, such as its sodium, potassium or its triethanol ammonium salt, and the sulphonic acid itself with the INCI name Phenylbenzimidazolesulphonic Acid (trade name: Eusolex 232).

For the purposes of the present invention, the preparations advantageously comprise the UV filter substances in a total amount of, for example, 0.1% by weight to 30% by weight, preferably 0.5 to 20% by weight, in particular 1.0 to 15.0% by weight, in each case based on the total weight of the preparations.

Particularly advantageous preparations for the purposes of the present invention comprise an emulsifier system which consists of
A. at least one emulsifier A chosen from the group of completely neutralized, partially neutralized or unneutralized, branched and/or unbranched, saturated and/or unsaturated fatty acids with a chain length of from 10 to 40 carbon atoms,
B. at least one emulsifier B chosen from the group of polyethoxylated fatty acid esters with a chain length of from 10 to 40 carbon atoms and with a degree of ethoxylation of from 5 to 100, and
C. at least one coemulsifier C chosen from the group of saturated and/or unsaturated, branched and/or unbranched fatty alcohols with a chain length of from 10 to 40 carbon atoms.

The emulsifier or emulsifiers A are preferably chosen from the group of fatty acids, which are completely or partially neutralized with customary alkalis (such as, for example, sodium hydroxide and/or potassium hydroxide, sodium carbonate and/or potassium carbonate, and mono- and/or triethanolamine). Stearic acid and stearates, isostearic acid and isostearates, palmitic acid and palmitates, and myristic acid and myristates, for example, are particularly advantageous.

The emulsifier or emulsifiers B are preferably chosen from the following group: PEG-9 stearate, PEG-8 distearate, PEG-20 stearate, PEG-8 stearate, PEG-8 oleate, PEG-25 glyceryl trioleate, PEG40 sorbitan lanolate, PEG-15 glyceryl ricinoleate, PEG-20 glyceryl stearate, PEG-20 glyceryl isostearate, PEG-20 glyceryl oleate, PEG-20 stearate, PEG-20 methylglucose sesquistearate, PEG-30 glyceryl isostearate, PEG-20 glyceryl laurate, PEG-30 stearate, PEG-30 glyceryl stearate, PEG40 stearate, PEG-30 glyceryl laurate, PEG-50 stearate, PEG-100 stearate, PEG-150 laurate. Polyethoxylated stearic esters, for example, are particularly advantageous.

According to the invention, the coemulsifier or the coemulsifiers C are preferably chosen from the following group: behenyl alcohol (C₂₂H₄₅OH), cetearyl alcohol [a mixture of cetyl alcohol (C₁₆H₃₃OH) and stearyl alcohol (C₁₈H₃₇OH)], lanolin alcohols (wool wax alcohols which are the unsaponifiable alcohol fraction of wool wax which is obtained following saponification of wool wax). Cetyl and cetylstearyl alcohol are particularly preferred.

It is advantageous according to the invention to choose the weight ratios of emulsifier A to emulsifier B to coemulsifier C (A:B:C) as a:b:c, where a, b and c, independently of one another, may be rational numbers from 1 to 5, preferably from 1 to 3. A weight ratio of, for example, 1:1:1 is particularly preferred.

For the purposes of the present invention, it is advantageous to choose the total amount of the emulsifiers A and B and of coemulsifier C from the range from 2 to 20% by weight, advantageously from 5 to 15% by weight, in particular from 7 to 13% by weight, in each case based on the total weight of the formulation.

It may be advantageous, although not necessary, for the formulations according to the present invention to comprise further emulsifiers. Preference is given to using those emulsifiers which are suitable for the preparation of W/O emulsions, it being possible for these to be present either individually or in any combinations with one another.

Preferably, for the purposes of the present invention, the further emulsifier(s) is/are chosen from the group of hydrophilic emulsifiers. According to the invention, particular preference is given to mono-, di- and tri-fatty acid esters of sorbitol.

The total amount of further emulsifiers is, according to the invention, advantageously chosen to be less than 5% by weight, based on the total weight of the formulation.

The list of given further emulsifiers which can be used for the purposes of the present invention is not of course intended to be limiting.

Particularly advantageous preparations for the purposes of the present invention are free from mono- or diglyceryl fatty acid esters. Particular preference is given to preparations according to the invention which comprise no glyceryl stearate, glyceryl isostearate, glyceryl diisostearate, glyceryl oleate, glyceryl palmitate, glyceryl myristate, glyceryl lanolate and/or glyceryl laurate.

The oil phase of the preparations according to the invention is advantageously chosen from the group of polar lipids with a polarity of ≦35 mN/m. Particularly advantageous lipids for the purposes of the present invention are all native lipids, such as, for example, olive oil, sunflower oil, soybean oil, peanut oil, rapeseed oil, almond oil, palm oil, coconut oil, castor oil, wheat germ oil, grape seed oil, thistle oil, evening primrose oil, macadamia nut oil, corn seed oil, avocado oil and the like, and those listed below.

| | | | Polarity |
|---|---|---|---|
| Manufacturer | Trade name | INCI name | mN/m |
| Cognis | Cetiol OE | Dicaprylyl Carbonate | 31.7 |
| Cognis | Cetiol CC | Dicaprylyl Ether | 30.9 |
| Stearinerie Dubois Fils | DUB VCI 10 | Isodecyl Neopentanoate | 29.9 |
| Lipo Chemicals Inc | Liponate TDTM | Tridecyl Trimellitate | 27.2 |
| Wacker | Wacker AK 100 | Dimethicone | 26.9 |
| Henkel Cognis | Eutanol G | Octyldodecanol | 24.8 |
| | Macadamia Nut Oil | | 22.1 |
| Bayer AG, | Silikonol VP 1120 | Phenyl Trimethicone | 22.7 |
| Dow Corning | | | |
| Henkel Cognis | Isopropylstearat | Isopropyl Stearate | 21.9 |
| WITCO, | Finsolv TN | C12-15 Alkyl Benzoate | 21.8 |
| Goldschmidt | | | |
| Dr. Straetmans | Dermofeel BGC | Butylene Glycol Dicaprylate/Dicaprate | 21.5 |
| Unichema | Miglyol 812 | Caprylic/Capric Triglyceride | 21.3 |
| Huels | | | |
| Henkel Cognis | Cetiol B | Dibutyl Adipate | 14.3 |
| Condea Augusta S.P.A. | Cosmacol ELI | C12-13 Alkyl Lactate | 8.8 |
| Condea Augusta S.P.A. | Cosmacol ETI | Di-C12/13 Alkyl Tartrate | 7.1 |
| Henkel Cognis | Myritol 331 | Cocoglycerides | 5.1 |
| Symrise | Corapan TQ | Diethylhexyl Naphthalate | n.b. |

It is very particularly advantageous for the purposes of the present invention to use one or more UV filter substances for the foam-boosting of self-foaming, foam-like, after-foaming or foamable cosmetic and dermatological preparations which comprise butylene glycol caprylate/caprate, C12-15 alkyl benzoate and/or Di-C12/13 alkyl tartrate.

Of the hydrocarbons, paraffin oil and further hydrogenated polyolefins, such as hydrogenated polyisobutenes, squalane and squalene, are to be used advantageously for the purposes of the present invention.

The content of lipids is advantageously chosen to be less than 50% by weight, preferably between 1 and 40% by weight, particularly preferably between 5 and 15% by weight, in each case based on the total weight of the foamable preparation.

If the lipid phase comprises oil-soluble UV filter substances, it is advantageous to choose the content of the lipid phase to be less than 80% by weight, preferably between 1 and 40% by weight, particularly preferably between 5 and 30% by weight, in each case based on the total weight of the foamable preparation.

In some instances, it may be advantageous, but not obligatory, if the oil phase of the preparations also comprises nonpolar lipids for the purposes of the present invention.

The cosmetic and/or dermatological preparations according to the invention can have the customary composition. Particularly advantageous for the purposes of the present invention are skincare preparations: they can be used for cosmetic and/or dermatological light protection, and also for the treatment of the skin and/or of the hair and as a make-up product in decorative cosmetics. A further advantageous embodiment of the present invention consists in after-sun products.

Depending on their formulation, cosmetic or topical dermatological compositions for the purposes of the present invention can, for example, be used as skin protection cream, day or night cream etc. It is optionally possible and advantageous to use the compositions according to the invention as bases for pharmaceutical formulations.

Just as emulsions of liquid and solid consistency are used as cosmetic cleansing lotions or cleansing creams, the preparations according to the invention can also represent “cleansing foams”, which can be used, for example, for the removal of make-up or as a mild washing foam—optionally also for blemished skin. Such cleansing foams can also advantageously be applied as so-called “rinse off” preparations, which are rinsed off from the skin following application.

The cosmetic and/or dermatological preparations according to the invention can also advantageously be in the form of a foam for the care of hair and/or the scalp, in particular a foam for arranging the hair, a foam which is used while blow-drying the hair, i.e., a styling and treatment foam.

It is also advantageous for the purposes of the present invention to create cosmetic and dermatological preparations whose main purpose is not protection against sunlight but that nevertheless contain some UV protection substances. Thus, for example, UV-A and/or UV-B filter substances are usually incorporated into day creams or makeup products. UV-protection substances, like antioxidants and, if desired, preservatives, also represent effective protection of the preparations themselves against spoilage. Also favourable are dermatological preparations which are in the form of a sunscreen composition.

For use, the cosmetic and dermatological preparations according to the invention are applied to the skin and/or the hair in an adequate amount in the manner customary for cosmetics.

The cosmetic or dermatological compositions according to the invention can, for example, be removed from aerosol containers and thereby foamed. Aerosol containers according to the invention are spray devices with a filling of the liquid or slurry-like substances, which are under the pressure of a propellant (pressurized gas or aerosol packagings). Such containers can be provided with valves of very different construction which permit the removal of the contents in the form of a foam.

In addition, the preparations according to the present invention may advantageously be removed from propellant gas-free, mechanically operated pump atomizers (pump dispensers). Particularly advantageous for the purposes of the present invention are pump systems which work without pressurized gas, but with a filter that brings about special swirling.

The cosmetic or dermatological preparations according to the invention can also preferably be removed, for example, from two-chamber aerosol containers and be applied to the skin. Packaging means advantageous according to the invention are containers in which there is one chamber with a filling of the liquid or slurry-like preparations under the pressure of a primary propellant located in a second chamber. Such containers can be equipped with valves of very different construction which permit the removal of the contents of the first chamber in the form of an emulsion or gel in any position—even with the valve pointing downward.

An advantageous embodiment is the BiCan® aerosol container in which the product is enclosed in a flexible bag made of metal or plastic within the can.

After-foaming compositions according to the invention are unfoamed directly after exiting an aerosol container and are multiphase systems—generally emulsions. They can be made into foams by gentle rubbing, for example in the hands or during application and rubbing on the skin, but also by stirring or other foaming operations.

Moreover, it has surprisingly been found that in the case of the use of (secondary) propellants, particularly advantageously of propellants soluble in the optionally present oil phase—for example, customary propane/butane mixtures—the preparations according to the invention are not simply sprayed as aerosol droplets, but develop into finely-bubbled, rich foams as soon as such systems containing such (secondary) propellants experience decompression.

In the case of the use of hydrocarbons or mixtures thereof having 4 or 5 carbon atoms—in particular isobutane, n-pentane and isopentane—as (secondary) propellant, it is possible to delay the automatic foaming after exiting from the pressurized packaging.

As a result of the evaporation of the secondary propellant in the applied cosmetic product, heat is additionally withdrawn from the skin and a pleasant cooling effect is achieved. Such after-foaming preparations are therefore likewise regarded as advantageous embodiments of the present invention with independent inventive step.

Suitable pressurized gas containers for the purposes of the present invention are primarily cylindrical vessels made of metal (aluminium, tinplate, contents <1 000 ml), protected or shatter-resistant glass or plastic (contents <220 ml) or shattering glass or plastic (contents <150 ml), in the choice of which compressive strength and breaking strength, corrosion resistance, ease of filling, or ease of sterilizing etc., but also aesthetic aspects, handleability, printing properties etc. play a role. The maximum permissible operating pressure of spray cans made of metal at 50° C. is 12 bar and the maximum fill volume at this temperature is about 90% of the total volume. For glass and plastic cans, the values for the operating pressure are lower and dependent on the size of the container and the propellant (whether liquefied, compressed or dissolved gas).

For the purposes of the present invention, cans made of tinplate, aluminium and glass are particularly advantageous. For reasons of corrosion protection, metal cans can be coated on the inside (silver- or gold-coated), for which purpose all standard commercial internal protective coatings are suitable. For the purposes of the present invention, preference is given to polyester, epoxyphenol and polyamide-imide coatings. Film laminates made of polyethylene (PE), polypropylene (PP) and/or polyethylene terephthalate (PET) on the inside of the cans are also advantageous, in particular for cans made of tinplate.

The pressurized gas containers are often single-part or two-part, but in most cases three-part cylindrical, conical or differently shaped. If plastics are used as the spray container material, then these should be resistant to chemicals and the sterilization temperature, gas-tight, impact-resistant and stable to internal pressures in excess of 12 bar. In principle, polyacetals and polyamides are suitable for spray container purposes.

The internal construction of the spray cans and the valve construction are many and varied, depending on the intended use and the physical nature of the ingredient—e.g. whether it is in the form of a two-phase or three-phase system—and can be determined by the person skilled in the art by simple trial and error without inventive activity. For suitable variants, reference may be made to the “Aerosol Technologie Handbuch der Aerosol-Verpackung” [Aerosol Technology Handbook of Aerosol Packaging] (Wolfgang Tauscher, Melcher Verlag GmbH Heidelberg/Munich, 1996).

Valves which are advantageous according to the invention can be designed with or without riser tube. The individual components from which valves according to the invention are usually constructed preferably consist of the following materials:
Disk: tinplate: uncoated, gold- or clear-coated, film-laminated (PE, PP or PET) aluminium: uncoated, silver- or gold-coated, different coating variants, Stoner-Mudge design
Seal: natural or synthetic elastomers or thermoplastic (sleeve gaskets, film-laminated made of PE or PP) internal and external seals, e.g. made of perbunan, buna, neoprene, butyl, CLB, LDPE, viton, EPDM, chlorobutyl, bromobutyl and/or diverse compounds
Cone: PA, POM, brass and diverse special materials, standard bores (e.g.: 0.25 to 0.70 mm or 2×0.45 to 2×1.00 mm), various shaft diameters
Spring: metal, particularly preferably V2A, stainless steel; plastic and also elastomer
Casing: standard and impact VPH bores, RPT bores or slit for overhead applications materials: e.g. polyacetal, PA, PE, POM and the like
Riser tube: plastic (polymer resin), e.g. PE, PP, PA or polycarbonate

Advantageous spray heads for the purposes of the present invention are, for example, foaming heads for upright use (hold can vertically) or foam heads for overhead application using one or more channels.

Suitable propellants are the customary “classic” readily volatile, liquefied propellant gases, such as, for example, dimethyl ether (DME) and/or linear or branched-chain hydrocarbons with two to five carbon atoms (such as, in particular, ethane, propane, butane, isobutane and/or pentane), which can be used on their own or in a mixture with one another.

Compressed air, and also other gases which are under pressure, such as air, oxygen, nitrogen, hydrogen, helium, krypton, xenon, radon, argon, nitrous oxide (N₂O) and carbon dioxide (CO₂), are also advantageously to be used for the purposes of the present invention as propellant gases (either on their own or in any desired mixtures with one another).

The cosmetic and dermatological preparations according to the invention can comprise cosmetic auxiliaries as are customarily used in such preparations, e.g. preservatives, preservative aids, complexing agents, bactericides, perfumes, substances for preventing or increasing foaming, dyes, pigments which have a colouring action, thickeners, moisturizing and/or humectant substances, fillers which improve the feel on the skin, fats, oils, waxes or other customary constituents of a cosmetic or dermatological formulation, such as alcohols, polyols, polymers, foam stabilizers, electrolytes, organic solvents or silicone derivatives.

Advantageous preservatives for the purposes of the present invention are, for example, formaldehyde donors (such as, for example, DMDM hydantoin, which is available, for example, under the trade name Glydant™ from Lonza), iodopropyl butylcarbamates (such as those available under the trade names Glycacil-L and Glycacil-S, both from Lonza, and/or Dekaben LMB from Jan Dekker), parabens (i.e. alkyl p-hydroxybenzoates, such as methyl-, ethyl-, propyl- and/or butylparaben), phenoxyethanol, ethanol, benzoic acid and the like. In addition, the preservative system according to the invention also usually advantageously comprises preservative aids, such as, for example, ethylhexylglycerol, glycine soya, etc.

Advantageous complexing agents for the purposes of the present invention are, for example, EDTA, [S,S]-ethylenediamine disuccinate (EDDS), which is available, for example, under the trade name Octaquest from Octel, pentasodium ethylenediamine tetramethylenephosphonate, which is available, for example, under the trade name Dequest 2046 from Monsanto and/or iminodisuccinic acid, which is available, inter alia, from Bayer AG under the trade names Iminodisuccinate VP OC 370 (about 30% strength solution) and Baypure CX 100 solid.

Particularly advantageous preparations are also obtained when antioxidants are used as additives or active ingredients. According to the invention, the preparations advantageously comprise one or more antioxidants. Favourable, but nevertheless optional, antioxidants which may be used are all antioxidants customary or suitable for cosmetic and/or dermatological applications.

For the purposes of the present invention, water-soluble antioxidants may be used particularly advantageously, such as, for example, vitamins, e.g. ascorbic acid and derivatives thereof.

Preferred antioxidants are also vitamin E and derivatives thereof, and vitamin A and derivatives thereof.

The amount of antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or derivatives thereof are the antioxidant or the antioxidants, it is advantageous to choose their respective concentrations from the range from 0.001 to 10% by weight, based on the total weight of the formulation.

If vitamin A or vitamin A derivatives or carotenes or derivatives thereof are the antioxidant or the antioxidants, it is advantageous to choose their respective concentrations from the range from 0.001 to 10% by weight, based on the total weight of the formulation.

It is particularly advantageous when the cosmetic preparations according to the present invention comprise cosmetic or dermatological active ingredients, preferred active ingredients being antioxidants which can protect the skin against oxidative stress.

Further advantageous active ingredients for the purposes of the present invention are natural active ingredients and/or derivatives thereof, such as, for example, α-lipoic acid, phytoene, D-biotin, coenzyme Q10, α-glucosylrutin, carnitine, carnosine, natural and/or synthetic isoflavonoids, creatine, creatinine, taurine and/or β-alanine, and 8-hexadecene-1,16-dicarboxylic acid (dioic acid, CAS number 20701-68-2; provisional INCI name Octadecenedioic acid) and/or Licochalcone A, which is characterized by the following structural formula: 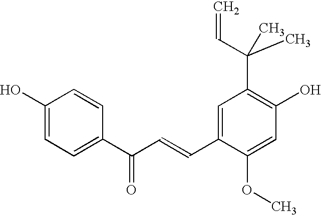
Licochalcone can advantageously also be used as a constituent of plant extracts, in particular from aqueous Radix Glycyrrhizae inflatae.

It is advantageous according to the invention when the cosmetic or dermatological preparations comprise 0.001 to 10% by weight, in particular 0.05 to 5% by weight, very particularly 0.01 to 2% by weight of an extract from Radix Glycyrrhizae inflatae, in each case based on the total weight of the preparation.

It is very particularly advantageous to start from an extract which is obtainable under the name Polyol Soluble Licorice Extract PU (INCI name Glycyrrhiza Inflata) from Maruzen. The extract from Radix Glycyrrhizae inflatae contains a content of about 25% Licochalcone A.

Formulations according to the invention which comprise, for example, known antiwrinkle active ingredients, such as flavone glycosides (in particular α-glucosylrutin), coenzyme Q10, vitamin E and/or derivatives and the like are particularly advantageously suitable for the prophylaxis and treatment of cosmetic or dermatological changes in the skin, as arise, for example, during skin aging (such as, for example, dryness, roughness and formation of dryness wrinkles, itching, reduced refatting (e.g. after washing), visible vascular dilations (telangiectases, cuperosis), flaccidity and formation of wrinkles and lines, local hyperpigmentation, hypopigmentation and incorrect pigmentation (e.g. age spots), increased susceptibility to mechanical stress (e.g. cracking) and the like). In addition, they are advantageously suitable to counter the appearance of dry or rough skin.

The water phase of the preparations according to the present invention can advantageously comprise customary cosmetic auxiliaries, such as, for example, alcohols, in particular those of low carbon number, preferably ethanol and/or isopropanol, diols or polyols of low carbon number, and ethers thereof, preferably propylene glycol, glycerol, butylene glycol, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilizers, electrolytes, and in particular one or more thickeners, which may advantageously be chosen from the group consisting of silicon dioxide, aluminium silicates or polysaccharides or derivatives thereof, e.g. hyaluronic acid, xanthan gum, hydroxypropylmethylcellulose, particularly advantageously from the group of polyacrylates, preferably a polyacrylate from the group of so-called Carbopols [from Bf. Goodrich], for example carbopol grades 980, 981, 1382, 2984, 5984, ETD 2020, ETD 2050, Ultrez 10, in each case individually or in combination.

In addition, the preparations according to the present invention can advantageously also comprise self-tanning substances, such as, for example, dihydroxyacetone and/or melanin derivatives in concentrations of from 1% by weight to 8% by weight, based on the total weight of the preparation.

In addition, the preparations according to the present invention can advantageously also comprise repellents for protection against flies, ticks and spiders and the like. For example, N,N-diethyl-3-methylbenzamide (trade name: meta-delphene, “DEET”), dimethyl phthalate (trade name: Palatinol M, DMP) and in particular ethyl 3-(N-n-butyl-N-acetylamino)propionate (available under the trade name lnsekt Repellent® 3535 from Merck) are advantageous. The repellents can either be used individually or in combination.

Moisturizers are substances or mixtures of substances that impart to cosmetic or dermatological preparations the property, following application or distribution on the surface of the skin, of reducing moisture release by the horny layer (also called trans-epidermal water loss (TEWL)) and/or of positively influencing hydration of the horny layer.

Advantageous moisturizers for the purposes of the present invention are, for example, glycerol, lactic acid and/or lactates, in particular sodium lactate, butylene glycol, propylene glycol, methylpropanediol, biosaccharide gum-1, glycine soya, ethylhexyloxyglycerol, pyrrolidonecarboxylic acid and urea. In addition, it is particularly advantageous to use polymeric moisturizers from the group of water-soluble and/or water-swellable and/or water-gellable polysaccharides. Hyaluronic acid, chitosan and/or a fucose-rich polysaccharide, which is filed in the Chemical Abstracts under the registry number 178463-23-5 and which is available, for example, under the name Fucogel® 1000 by SOLABIA S.A., for example, are particularly advantageous. Moisturizers can advantageously also be used as anti-wrinkle active ingredients for the prophylaxis and treatment of cosmetic or dermatological changes in the skin, as arise, for example, during skin aging.

The cosmetic or dermatological preparations according to the invention can also advantageously, but not necessarily, comprise fillers, which, for example, further improve the sensory and cosmetic properties of the formulations and, for example, bring about or enhance a velvety or silky feel on the skin. Advantageous fillers for the purposes of the present invention are starch and starch derivatives (such as, for example, tapioca starch, distarch phosphate, aluminium or sodium starch octenylsuccinate and the like), pigments which have neither primarily UV filter effect nor colouring effect (such as, for example, boron nitride etc.) and/or Aerosils® (CAS No. 7631-86-9).

The examples below serve to illustrate the present invention without limiting it. Unless stated otherwise, all amounts, proportions and percentages are based on the weight and the total amount or on the total weight of the preparations.

### EXAMPLES

Various samples were analysed with regard to their bubble count, bubble size, bubble fraction and bubble circumference.

| | Comparative | |
|---|---|---|
| | Example | Example 1 |
| | % by wt. | % by wt. |
| Stearic acid | 2.00 | 2.00 |
| Cetyl alcohol | 2.00 | 2.00 |
| PEG-40 stearate | 2.00 | 2.00 |
| Sorbitan stearate | 0.75 | 0.75 |
| Cetyl ricinolate | 1.00 | 1.00 |
| Butylene glycol | 8.00 | 8.00 |
| caprylate/caprate | | |
| Ethylhexyltriazine | | 2.00 |
| Cyclomethicone | 0.50 | 0.50 |
| Glycerol | 5.00 | 5.00 |
| Perfume, preservative, | q.s. | q.s. |
| dyes, etc. | | |
| Sodium hydroxide | q.s | q.s |
| Water | ad 100 | ad 100 |

pH adjusted to 5.0-6.0

To prepare the foam, 96% by volume of the emulsion are foamed with 4% by volume of isobutane/propane/butane.

The microscopic foam analysis revealed the following results:

| | Comparative | |
|---|---|---|
| | Example | Example 1 |
| Bubble number | 954 | 2143 |
| Bubble fraction (%) | 48 | 45 |
| Average bubble size (mm²) | 0.02 | 0.01 |
| Average circumference (mm) | 0.45 | 0.3 |
| Average distance apart | 16.8 | 11.4 |

As shown in FIGS. 1 and 2, it is clear that the bubble size in Example 1 is smaller than in the Comparative Example, whereas the bubble number in the analysed image section (i.e. in the same volume) is significantly greater than in the Comparative Example. This means that the preparation for the purposes of the present invention clearly has more bubbles and smaller bubbles than the preparation of the prior art. In addition, the fraction of bubbles (the gas fraction of the preparation) is less, thus resulting in relatively more emulsion for the same volume. This explains, inter alia, the more compact, more caring texture of the preparation for the purposes of the present invention.

### Example 2

| Emulsion | | A | B | C |
|---|---|---|---|---|
| Stearic acid | | 2 | 2 | |
| Palmitic acid | | | | 1.5 |
| Cetyl alcohol | | 2.5 | 2 | |
| Stearyl alcohol | | | | 3 |
| PEG-100 stearate | | | | 3.5 |
| PEG-40 stearate | | | 2 | |
| PEG-20 stearate | | 3 | | |
| Sorbitan stearate | | | 0.8 | |
| C₁₂₋₁₅ Alkyl benzoate | | 5 | | |
| C₁₂₋₁₃ Alkyl tartrate | | | | 7 |
| Butylene glycol dicaprylate/dicaprate | | | 6 | |
| Dicaprylyl ether | | | | 2 |
| Cyclomethicone | | | 2 | 3 |
| Butylene glycol | | 1 | | |
| Isohexadecane | | 2 | | |
| Methylpropanediol | | | | |
| Propylene glycol | | | | 5 |
| Glycerol | | 5 | 7 | |
| UVASorb ® K2A | | | | 2 |
| Uvinul A Plus ® | | 2 | | |
| NeoHeliopan ® AP | | | | |
| Phenylbenzimidazole sulphonic acid | | | | |
| Ethylhexyl methoxycinnamate | | | | |
| Ethylhexyltriazone | | 2 | 2 | 2 |
| Octocrylene | | 2 | | |
| Bis-Ethylhexyloxyphenol methoxy-phenyltriazine | | | 3 | 3 |
| Glycyrrhiza Inflata | | 0.05 | | |
| Creatine | | | | 1 |
| Creatinine | | | | 0.1 |
| Vitamin E acetate | | | 0.5 | |
| BHT | | | | 0.1 |
| Na₂H₂EDTA | | 0.50 | | |
| Perfume, preservative | | q.s. | q.s. | q.s. |
| Dyes, etc. | | q.s. | q.s. | q.s. |
| Sodium hydroxide | | q.s. | | q.s. |
| Potassium hydroxide | | | q.s. | |
| Water | | ad 100.0 | ad 100.0 | ad 100.0 |
| Emulsion | D | E | F | G |
| Stearic acid | 1.5 | | | |
| Palmitic acid | | | 3 | 3 |
| Cetyl alcohol | | 3 | | |
| Cetylstearyl alcohol | | | 2 | 2 |
| Stearyl alcohol | 3 | | | |
| PEG-100 stearate | | 4 | | |
| PEG-40 stearate | 3 | | | |
| PEG-20 stearate | | | 3 | 3 |
| Sorbitan stearate | 1 | | | |
| Tridecyl trimellitate | | 5 | | |
| C₁₂₋₁₅ Alkyl benzoate | | | 3 | 3 |
| Butylene glycol dicaprylate/dicaprate | 8 | | | |
| Octyldodecanol | | 2 | | |
| Cocoglycerides | | | | 2 |
| Dicaprylyl ether | | | 2 | 2 |
| Cyclomethicone | | | | |
| Dimethicone | 1 | | 2 | 2 |
| Isohexadecane | | 3 | | |
| Methylpropanediol | | 4 | | |
| Propylene glycol | | | | |
| Glycerol | 5 | | 6 | 6 |
| NeoHeliopan ® AP | | 2 | | |
| Phenylbenzimidazole sulphonic acid | 1 | 4 | 1 | 1 |
| Ethylhexyl methoxycinnamate | 5 | | 4 | 4 |
| Ethylhexyltriazone | | | | |
| Diethylhexylbutamidotriazone | 1 | | | |
| Butylmethoxydibenzoylmethane | 2.5 | | 2 | 2 |
| Bis-Ethylhexyloxyphenol methoxy- | 2 | | | |
| phenyltriazine | | | | |
| Licochalcone A | | | | 0.01 |
| Taurine | | 0.3 | | |
| Vitamin E acetate | 0.2 | | 0.3 | 0.3 |
| BHT | | 0.05 | | |
| Na₂H₂EDTA | | | 0.4 | 0.4 |
| Perfume, preservative | q.s. | q.s. | q.s. | q.s. |
| Dyes, etc. | q.s. | q.s. | q.s. | q.s. |
| Sodium hydroxide | q.s. | q.s. | q.s. | q.s. |
| Potassium hydroxide | | | | q.s. |
| Water | ad 100.0 | ad 100.0 | ad 100.0 | ad 100.0 |

To prepare the (aerosol) foam, 80-97% by volume of the emulsion (A-G) are in each case foamed with 3-20% by volume of a suitable gas (e.g. propane/butane, compressed air, nitrogen).

1. A method for boosting the foam of a cosmetic or dermatological preparation comprising adding one or more UV filter substances to a self-foaming, foam-like, after-foaming or foamable cosmetic or dermatological preparation.

2. The method of claim 1, wherein the preparation comprises at least one polar oil.

3. The method of claim 1, wherein the one or more UV filter substances include at least one UV filter substance selected from the group consisting of:
2,4,6-tris[anilino(p-carbo-2′-ethyl-1′-hexyloxy)]-1,3,5-triazine,
2-ethylhexyl 4-methoxycinnamate,
4-(tert-butyl)-4′-methoxydibenzoylmethane,
2,4-bis{[4-(2-ethylhexyloxy)-2-hydroxy]phenyl}-6-(4-methoxyphenyl)-1,3,5-triazine, and
2-phenylbenzimidazole-5-sulphonic acid and salts thereof.

4. The method of claim 3, wherein the preparation comprises at least one polar oil.

5. The method of claim 1, further comprising the step of applying the preparation to the skin prior to exposing the skin to sunlight.

6. The method of claim 1, further comprising the step of treating the effects of the sun on the skin by applying the preparation to the skin.

7. A method for reducing the bubble size of a cosmetic or dermatological preparation comprising adding one or more UV filter substances to a self-foaming, foam-like, after-foaming or foamable preparation.

8. The method of claim 7, wherein the preparation comprises at least one polar oil.

9. The method of claim 7, wherein after said adding step the preparation has an average bubble size of from 0.005 to 0.5 mm².

10. The method of claim 7, wherein the one or more UV filter substances include at least one UV filter substance selected from the group consisting of:
2,4,6-tris[anilino(p-carbo-2′-ethyl-1′-hexyloxy)]-1,3,5-triazine,
2-ethylhexyl 4-methoxycinnamate,
4-(tert-butyl)-4′-methoxydibenzoylmethane,
2,4-bis{[4-(2-ethylhexyloxy)-2-hydroxy]phenyl}-6-(4-methoxyphenyl)-1,3,5-triazine, and
2-phenylbenzimidazole-5-sulphonic acid and salts thereof.

11. The method of claim 10, wherein the preparation comprises at least one polar oil.

12. The method of claim 7, further comprising the step of applying the preparation to the skin prior to exposing the skin to sunlight.

13. The method of claim 7, further comprising the step of treating the effects of the sun on the skin by applying the preparation to the skin.

14. A self-foaming, foam-like, after-foaming or foamable dermatological or cosmetic preparation comprising at least one UV filter substance selected from the group consisting of:
2,4,6-tris[aniline(p-carbo-2′ethyl-1′-hexyloxy)]-1,3,5-triazine,
2-ethylhexyl 4-methoxycinnamate,
4-(tert-butyl)-4′-methoxydibenzoylmethane,
2,4-bis{[4-(2-ethylhexyloxy)-2-hydroxy]phenyl}-6-(4-methoxyphenyl)-1,3,5-triazine, and
2-phenylbenzimidazole-5-sulphonic acid and salts thereof;
wherein said UV filter substance boosts the foam of the self-foaming, foam-like, after-foaming or foamable dermatological or cosmetic preparation compared to preparations that do not include the at least one UV filter substance.

15. The dermatological or cosmetic preparation of claim 14, further comprising an emulsifier system comprising
at least one emulsifier A selected from the group consisting of completely neutralized, partially neutralized or unneutralized, branched or unbranched, saturated or unsaturated fatty acids having a chain length of from 10 to 40 carbon atoms
at least one emulsifier B selected from the group consisting of polyethoxylated fatty acid esters having a chain length of from 10 to 40 carbon atoms and with a degree of ethoxylation of from 5 to 100, and
at least one coemulsifier C selected from the group consisting of saturated or unsaturated, branched or unbranched fatty alcohols having a chain length of from 10 to 40 carbon atoms.

16. The dermatological or cosmetic preparation of claim 14, further comprising at least one polar oil.

17. The dermatological or cosmetic preparation of claim 16, wherein the at least one polar oil component comprises at least one polar oil having a polarity of less than or equal to 35 mN/m.

18. The dermatological or cosmetic preparation of claim 14, wherein said preparation has an average bubble size of from 0.005 to 0.5 mm² when foamed.

19. The dermatological or cosmetic preparation of claim 14, wherein the preparation is free of oil.

20. The dermatological or cosmetic preparation of claim 14, wherein the at least one UV filter substance is present in a total amount of from 1.0 to 15.0% by weight based on the total weight of the preparation.
